(19) United States
(12) Patent Application Publication
(10) Pub. No.: US 2002/0042600 A1
(43) Pub. Date: Apr. 11, 2002

(54) Full wrapping disposable refastenable and adjustable pant

(76) Inventors: Paul Joseph Datta, Appleton, WI (US); Suzanne Marie Schmoker, Oshkosh, WI (US)

Correspondence Address: BRINKS HOFER GILSON & LIONE, P.O. BOX 10395, CHICAGO, IL, 60610, US

(21) Appl. No.: 09/872,976

(22) Filed: May 31, 2001

Related U.S. Application Data
(63) Continuation of application No. 09/215,865, filed on Dec. 18, 1998, now abandoned.

(51) Int. Cl. A61F 13/15; A61F 13/20

(52) U.S. Cl. 604/385.13; 604/385.01; 604/385.3; 604/392

(57) ABSTRACT

A disposable pant for adults, toddlers and infants is provided. The pant has elastic side panels of sufficient width to be able to wrap around the pant when loaded with bodily wastes so as to form an elastic pouch for disposal. In an embodiment of the pant there is provided a pant having a body having a front end, a back end, and a crotch area. The body was a chassis, a front fastening panel, and two elastic side panels.

### BACKGROUND

This is a continuation of pending application Ser. No. 09/215,865, lawyer docket no. 659/487 (filed Dec. 18, 1998), this application further claims benefit of priority to Ser. No. 09/215,866 lawyer docket no. 659/482 (filed Dec. 18, 1998) and Ser. No. 09/215,951 lawyer docket no. 659/489 (filed Dec. 18, 1998), the entire disclosures of which are hereby incorporated by reference.

The field of disposable apparel for holding bodily fluids, such as incontinence type products is well established. There are a wide variety of products in this field, which have many uses including uses for adults, children and women. Some of these product types include those disclosed in Van Gompel et al. U.S. Pat. No. 4,940,464, Strohbeen et al. U.S. Pat. No. 4,610,681, Heran et al., U.S. Pat. No. 4,646,362, and Enloe U.S. Pat. No. 4,895,568.

### SUMMARY

In an embodiment of this invention there is provided a pant comprising a chassis and an elastic panel; the pant having a front and back end; the chassis comprising an absorbent core, a backing member and a bodyside liner; the chassis having a length and a width and a front and a back end; the elastic panel having a width and a length; the elastic panel adjacent the chassis and toward the back end of the chassis; the width of an elastic panel being such that the panel when stretched can encircle the pant when used. This pant may have a panel wide enough to encircle the used pant at least 1½ times.

In another embodiment of the invention there is provided an incontinence product for adults comprising: refastenable elastic side panels; the side panels having a length and a width; the incontinence product having a length and a width, the width of an elastic side panel being at least long enough to surround the product when used and fasten to itself, thereby forming an elastic disposable pouch. This product can hold at least about 100 grams of bodily waste. This product may further hold at least about 400 grams of bodily waste.

In yet a further embodiment there is provided a pant product for toddlers and infants comprising: refastenable elastic side panels; the side panels having a length and a width; the product having a length and a width, the width of an elastic side panel being at least long enough to fully surround the product when used and fasten to itself, thereby forming an elastic disposable pouch. This product when used may hold at least about 25 grams of bodily waste. This product when used may further hold at least about 100 grams of bodily waste.

In still another embodiment there is provided a pant product comprising a chassis and one elastic back panel; where the back panel can be separated into a left side portion and a right side portion by the centerline of the product; and each side portion having a length and a width; the product having a length and a width; the width of one of the elastic side portions being at least long enough to encircle the product when used and fasten to itself, thereby forming an elastic disposable pouch.

### DRAWINGS

FIG. 1A is a plan view of a pant showing the bodyside of the pant.

FIG. 1B is a plan view of a pant showing the outside of the pant.

FIGS. 1C to 1E are cross sectional views of the pant shown in FIGS. 1A and 1B.

FIG. 2 is a drawing of a pant being worn.

FIG. 3 is a plan view of a pant showing the bodyside of the pant.

### DETAILED DESCRIPTION OF PREFERRED EMBODIMENTS OF THE INVENTION

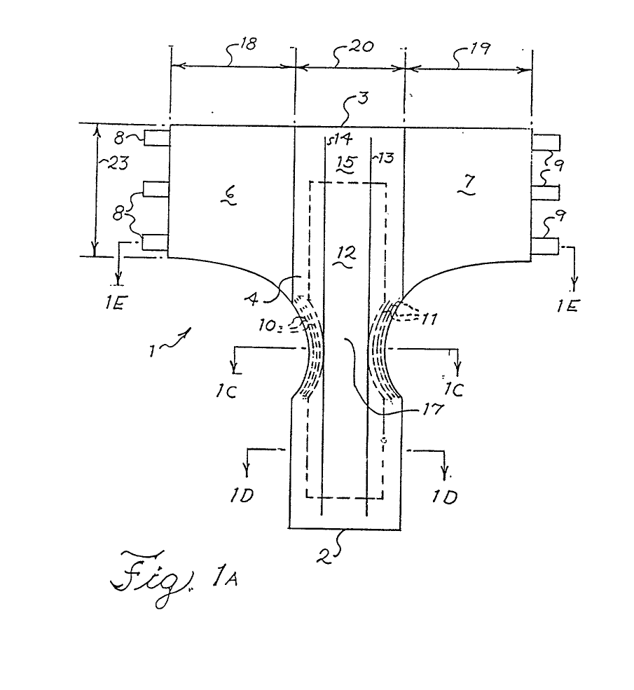

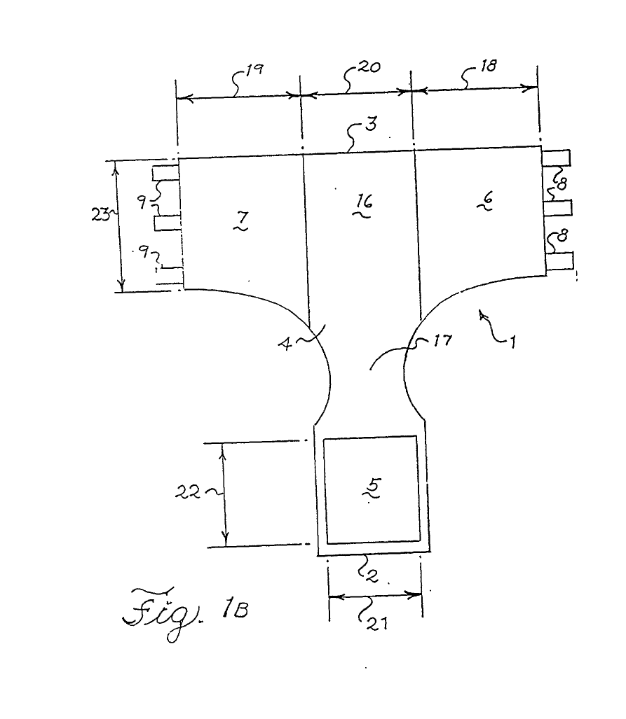

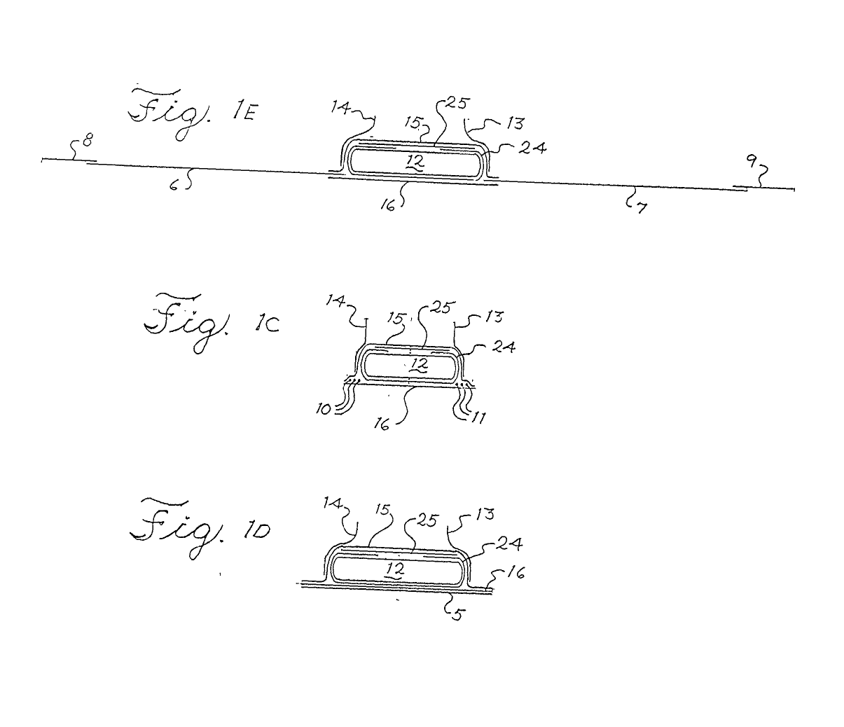

A construction of one embodiment of the pant is generally shown in FIGS. 1A and 113, with FIG. 1A showing the bodyside or inside of the pant and FIG. 1B showing the outside of the pant. Referring to these figures, the pant consists of a body 1 having a front end 2, a back end 3, and a crotch 2 area 17. The body has a chassis 4, a front fastening panel 5, and two elastic side panels 6 and 7. On each of the elastic side panels there are positioned closure devices 8 and 9 respectively. In these Figures, three such devices are shown for each side panel. Fewer or more closure devices may also be used. In the chassis, there may be positioned crotch elastics 10 and 11, an absorbent core or pad 12 and cuffs 13 and 14. On the bodyside of the chassis there may be a bodyside liner sheet 15, which may cover the absorbent pad. The relationship of the size of the elastic panels, the chassis and the front fastening panel, as well as the number and position of the closure devices, provides the ability to have a single product conformably and securely fit a wide range of sizes, body shapes and age groups (e.g. infants, toddlers and adults). As discussed herein unless specifically mentioned otherwise, when referring to the dimensions and size of elasticized components of the pant, these dimensions and sizes will be for the actual size of the component, i.e., the maximum size under tension, and not for the size of the component when tension is relaxed, i.e., the smaller size as the elastics contract.

Generally, the total width (the sum of 18 and 19) of the elastic side panels 6 and 7 should be about 20% or more of the width 20 of the chassis 4. Optimally, the total width of the elastic side panels should be about 50% or more of the width of the chassis and ideally should range from about 75% to about 500% of the width of the chassis.

Generally, the width 21 of the fastening panel 5 should be less than about 5 times (2 times when extended) the total width (the sum of 18 and 19) of the relaxed elastic side panels. Optimally, the width of the fastening panel should be less than about 4 times (2 times when under tension) the total width of the relaxed elastic side panels. Ideally, the width of the fastening panel should be less than about 2 times the total width of the relaxed elastic side panels. Alternatively, an outer member may serve as the fastening panel.

Generally, the length 22 of the fastening panel is at least about 15% of the length of the pant and ideally the length of the fastening panel is at least about 20% of the length of the pant. Generally, the combined length of the 3 elastic panels and the front fastening panel (the sum of 22 and 23) should be at least about 7% of the length of the total pant. Optimally, this combined length should be at least about 25% of the total pant and ideally this combined length should be from about 40% to about 60% of the length of the pant.

Generally, the width of the front and back ends of the chassis are the same. However, the chassis may be wider in front or back.

Generally, the optimal size relationship for the components of the pant are: for the width of the chassis and the front fastening panel to be about the same; for the width of the elastic side panels (the sum of 18 and 19) to be from about 100% to about 500% of the width of the chassis; for the pant to have three closure devices on each elastic panel; for the length of the front fastening panel to be from about 15% to about 30% of the total length of the pant and for the length of the elastic panels to be from about 15% to about 40% of the total length of the pant. This optimal size relationship may vary depending upon the elasticity of the side panels, their shape, their strength, and the shape and size of the chassis.

Cross sections of the pant are shown in FIG. 1C, taken along line C-C of FIG. 1A, FIG. 1D, taken along line D-D of FIG. 1A and FIG. 1E, taken along line E-E of FIG. 1A. Referring to these figures, the chassis 4 of the pant generally has an absorbent pad 12, which may be surrounded by a porous wrap 24. A surge layer 25 may also be positioned on the wrap. The pad and surge layers may be covered by the bodyside liner 15 and the backing member 16. The bodyside liner and backing member also cover the crotch elastics 10 and 11. Cuffs 13 and 14 are positioned on the bodyside liner of the pant and may have elastics. The elastic side panels 6 and 7 are attached to the chassis and the closure devices 8 and 9 are attached to the elastic side panels. The front fastening panel 5 is attached to the outer surface of the chassis.

Generally, the elastic side panels can be attached to the chassis by any suitable technique known to the art. The elastic side panels can be positioned between the backing member and the bodyside liner, on the bodyside of the bodyside liner or on the outside of the backing member. Optimally, they are positioned in-between the backing member and the bodyside liner. The elastic side panels and the chassis can overlap to greater or lesser extents depending upon the overall size of the pant, the length of the elastic panel, the strength of the components, the type of attachment used (i.e., glue, ultrasonic, etc.) and the costs associated with those components. By way of illustration, if the elastic side panels are attached to the outside of the chassis, they could overlap the chassis by about 5% to about 25% or more of the chassis width. When the elastic side panels are positioned between the bodyside liner and the backing member they should overlap these layers sufficiently for the manner of attachment to be effective. By way of illustration, and without any limitation on the size of the product, in a product for an adult having an overall width (the sum of 18, 19 and 20) of about 27 inches (686 mm), 36 inches in state under tension, 914 mm) and a chassis width 20 of about 13½ inches (343 mm), the overlap should optimally be about from ¼ of an inch (6.4 mm) to about 1½ inches (38 mm).

The elastic side panels can be attached to the chassis by the use of adhesives, ultrasonic bonding, heat, pressure, or any other technique known to the art. Generally, any type of adhesive that can provide a strong bond can be used. These would include by way of illustration hot melt adhesives, solvent-based adhesives, pressure sensitive adhesives, elastic attachment adhesives, and thermoset adhesives. Ideally, a hot melt adhesive may be used. By way of illustration, and with out any limitation on the types of adhesive that can be used, suitable hot melt adhesives can be obtained from: Ato-Findley Adhesives located in Wauwatosa, Wis. under the trade names H-2525A or H2096; and National Starch & Chemical Corporation located in Bridgewater, N.J. under the trade name 34-5610.

The adhesives may be applied through any number of techniques and in any number of patterns known to the art, which include by way of example and without limitation, spray, meltblown, slot coat, roll coat, gravure, spiral, rows, strips, or dots.

Ultrasonic bonding of the components can be accomplished by using any ultrasonic bonding device known to the art. Such devices can be obtained for example from Branson or Dukane. The pattern for the ultrasonic bond or weld should be such that it permits a good attachment of the components without adding unnecessarily to the stiffness of the pant, although as described herein in at least one embodiment having some stiffness in these bonds is desirable. Such patterns include by way of example and without limitation, staggered dots, aligned dots, bars, crescents, or combinations of these. The pattern can be achieved either by plunge bonding or rotary ultrasonic bonding.

Ideally, combinations of ultrasonic bonding and adhesives may be used. When using such combinations, the width of the adhesive zone and the width of the ultrasonic zone may vary or be the same. That is, the width of the adhesive zone may be larger than the width of the ultrasonic zone, smaller than the width of the ultrasonic zone, or the same size. Thus, again turning to the above illustrative example of an adult 27 inch (686 mm) (36 inches under tension, 914 mm) width product, and without any limitation, the adhesive zone would optimally coincide or be about ¼ inch (6.4 mm) narrower than the ultrasonic bonding zone.

The closure devices may be attached to the elastic panels on the bodyside, on the outside, or if the elastic panels are of a multilayer construction, in-between the layers of the elastic panels. The closure devices may be adhered to the elastic panels by any of the techniques and with any of the materials described herein for the attachment of one component to another, or by any other suitable fastening technique or material. Similarly, the amount of overlap between the elastic panels and the closure devices may vary. Additionally, some closure devices are equipped with adhesive on them and can thus be directly affixed to the elastic panel without the need for an additional adhesive or gluing step. Continuing with the above illustration of a 27 inch (686 mm) (36 inches under tension, 914 mm) wide adult pant, by way of example and without limitation, the closure devices are attached to the outside of the panel, overlap the panel by about 1 inch (25 mm), and are adhesively and ultrasonically bonded to the panel using a staggered dot pattern.

A front fastening panel may be attached onto the outside of the chassis. It may be the same size as the front section of the chassis, it may be smaller than the front section of the chassis, either width wise, length wise, or both, or it may be larger than the front section of the chassis. Optimally, the panel will be slightly smaller (about 1/16 of an inch (1.6 mm) to about 3 inches (76 mm)) in the width and length than the front section of the chassis. Moreover, the front panel may be any shape. The front panel may be adhered to the chassis by any techniques and materials described herein or by any other suitable fastening technique or material. Breathability of the pant may dictate the techniques or materials used when attaching the front panel to the chassis. Again, continuing with the example of a 27 inch (686 mm) (36 inches under tension, 914 mm) width pant, and without limitation, the front of the chassis would be about 13½ inches (343 mm) wide and the front fastening panel would be about 12½ inches (318 mm) wide.

The cuffs may be attached to the inside of the chassis by any of the techniques described herein or by another suitable fastening technique or material. The cuffs may be attached to the bodyside liner, the backing member, or some other layer of the chassis depending upon the width and position of these layers in the chassis. The cuffs may be positioned entirely over the absorbent pad, partially over the absorbent pad or entirely off the absorbent pad. Using the illustrative 27 inch (686 mm) (36 inches under tension, 914 mm) adult pant described above and without limitation, the cuffs would be located as shown in FIG. 1A, adjacent the absorbent pad. Examples of cuffs that may be used are disclosed in Enloe, U.S. Pat. No. 5,415,644, Enloe, U.S. Pat. No. 5,413,570, Enloe et al., U.S. Pat. No. 4,846,825, Enloe, U.S. Pat. No. 4,846,823, Enloe U.S. Pat. No. 4,704,116, Enloe U.S. Pat. No. 5,599,338, Schleinz, U.S. Pat. No. 5,649,918, Faulks WO 97/21410, and Le Maheiu et al., U.S. Pat. No. 5,620,431, all of which are assigned to Kimberly-Clark and the disclosures of which are herein incorporated by reference.

The elastic side panel may be made from any material that has elastic properties. It may be single layer or have multiple layers. For example, it 7 may be made from a multilayered material consisting of elastic filaments, fibers, sheets, ribbon or strands sandwiched between two layers of fabric. It may be made from material such as by way of example, the types of materials disclosed in Wideman U.S. Pat. No. 4,606,964 and Johnson U.S. Pat. No. 3,371,668, the disclosures of which are herein incorporated by reference. It may be made from a stretch bonded laminate type material, which consists of two layers of material and an elastomeric web that is melt blown or extruded as filaments between these two layers. It can also be a neck bonded type material or neckbonded-laminate (NBL). The two layers can be, by way of example and without limitation, spunbonded material, such as spunbonded polypropylene, or they may be rayon or a woven fabric type material, or film or apertured film. They may be breathable or non-breathable, although for use as elastic side panels it is preferable that the material be breathable. The elastomeric web may be made from Kraton®.

A further example of a type of material that may be used for the elastic side panels is known as “stretch bonded laminate” or SBL composite materials. These materials generally have at least two layers in which one layer is a gatherable layer and the other layer is an elastic layer. The layers are joined together when the elastic layer is in an extended condition so that upon relaxing the layers, the gatherable layer is gathered. Such a multilayer composite elastic material may be stretched to the extent that the nonelastic material gathered between the bond locations allow the elastic material to elongate. One type of multilayer composite elastic material is disclosed, for example, by U.S. Pat. No. 4,720,415 to Vander Wielen et al., the disclosure of which is herein incorporated by reference, and in which multiple layers of the same polymer produced from multiple banks of extruders are used. Other composite elastic materials are disclosed in U.S. Pat. No. 4,789,699 to Kieffer et al., U.S. Pat. No. 4,781,966 to Taylor and U.S. Pat. No. 4,657,802 and 4,652,487 to Morman and U.S. Pat. Nos. 4,655,760 and 4,692,371 to Morman et al., the disclosure of which are herein incorporated by reference.

Examples of types of NBL materials that may be used for the elastic panels are described in U.S. Pat. No. 5,226,992 issued Jul 13, 1993 to Morman; U.S. Pat. No. 5,336,545 issued Aug 9, 1994 to Morman; and U.S. Pat. No. 5,514,470 issued May 7, 1996 to Haffner et al., the disclosures of which are incorporated herein by reference.

An ideal material for the elastic side panels is a material that is breathable and consists of two layers of spunbonded polypropylene that have elastic filaments glued between them. This elastic side panel has elastic filaments that are glued between two layers of spunbonded polypropylene. The elastic filaments extend in the width wise dimension of the pant and panel. They are not parallel, but instead move closer together towards the end of the panel in what could be characterized as a zig-zag pattern. Ideally these elastic filament should consist of a single filament that was placed in the zig-zag pattern. Alternatively, however, multiple strands of elastic may be used, including parallel strands. These elastic filaments are held in place between the layers of spunbonded by the use of adhesives and/or ultrasonics. The adhesive may be any of the types disclosed herein, or known to the art that has elastic properties when set, such adhesives would include, by way of example and without limitation, National Starch & Chemical Company, Bridgewater, N.J., and Ato-Findley Adhesives, Wauwatosa, Wis. The adhesives may be applied by any technique known to the art, including those described herein.

An optimal way to apply the adhesives to form the elastic side panels, and one which gives rise to a new and different elastic material is to use a slot coat technique that provides for spaced apart parallel glue lines that are positioned length wise on the panel and the pant. Additionally, the transverse gathers may be formed by the use of ultrasonic bonding, heat or pressure.

These glue lines may be from about 1/32 of an inch (0.8 mm) or smaller in width to about 3½ inches (91 mm) in width or larger, and ideally range from about ⅛ inch (3.2 mm) in width to about 2 inches (51 mm) in width. The spacing between them may be the same as the width of the glue line or it may be different. Additionally, the glue lines may have varying widths. An ideal arrangement is for the glue lines to have a width of ⅛ inch (3.2 mm) and for the space between each glue line to also be ⅛ inch (3.2 mm). This spacing creates a material that has transverse gathers or pleats. It has been found that these transverse gathers greatly improve the comfort and fit of the pant and panel as well as increase the elastic range of the panel. That is, panels constructed with these transverse elastic gathers show a greater percentage elongation for the same tension and size of elastic filament than a panel that does not have these transverse elastic gathers. Depending on the type of adhesive and the amount used, the glue lines may result in attached zones in which the composite has reduced elasticity, regardless of the elasticity of the elastic, i.e., a zone of little or no elasticity in the panel. Alternatively, ultrasonic bond lines can replace the glue lines. These types of material are the subject of a separate patent application, which was filed on the same date as this application in the name of Fell et al., having lawyer identification number 659/489, which application is incorporated herein by reference.

The elastic panels made with these glue lines have a much higher maximum stretch than panels made by conventional techniques. For example, a panel made using ⅛ inch (3.2 mm) glue lines, spaced apart by ⅛ inch (3.2 mm) and having the elastic strands placed under 250% elongation will have a maximum elongation length of 240%. Under the same condition, it would be expected that the conventional composition would have a maximum elongated length of about 212%. Thus, these elastic panels allow for a much greater use of the tension put into the strands and provides a panel that for the same initial elastic elongation can have a substantially larger maximum width.

Examples of materials suitable for constructing the elastic side panels include elongatable materials, elastic materials, or elastomeric materials, such as polymer films, woven fabrics, knits, nonwoven fabrics or the like, as well as combinations thereof. Thus, the elastic side panels may be composed of a stretch-bonded-laminate (SBL) material, a neck-bonded-laminate (NBL) material, a reversibly necked nonwoven material, an elastomeric film, and elastomeric foam material, elastic threads or the like. For example, suitable meltblown elastomeric fibrous webs for forming elastic side panels are described in U.S. Pat. No. 4,663,220 issued May 5, 1987 to T. Wisneski et al., the disclosure of which is hereby incorporated by reference. Examples of composite fabrics comprising at least one layer of nonwoven textile fabric secured to a fibrous elastic layer are described in European Patent Application EP No. 0 217 032 A2 published on Apr. 8, 1987 with the inventors listed as J. Taylor et al., the disclosure of which is hereby incorporated by reference.

As described previously, the elastic side panels may be formed of a material capable of stretching in one direction or less desirably capable of stretching in multiple directions. One suitable one-directional stretch material is disclosed in U.S. Pat. No. 4,7820,415 issued Jan. 19, 1988 to Vander Wielen et al., which is incorporated herein by reference. The one-directional stretch material may comprise a composite material including at least one gatherable web bonded to at least one elongated elastic web. The elastic web may be an elastic film or nonwoven fibrous elastic webs such as meltblown elastomeric fibrous webs. In one embodiment, the expansion panels comprise a stretch bonded laminate formed of a prestretched elastic meltblown inner layer sandwiched between and attached to a pair of spunbond polypropylene nonwoven webs having a basis weight of about 13.6 gsm (0.4 oz/yd²). Suitable elastic materials can be purchased from the Shell Chemical Company of Houston, Tex. under the trade name KRATON.® Other suitable one-directional stretch materials are disclosed in U.S. Pat. No. 4,965,112 issued Oct. 23, 1990 to Morman; U.S. Pat. No. 4,606,964 issued Aug. 19, 1986 to Wideman; and U.S. Pat. No. 4,657,802 issued Apr. 14, 1987 to Morman.

Examples of two-directional stretch materials that could be used for the elastic side panels are disclosed in U.S. Pat. No. 5,114,781 issued May 19, 1992 and U.S. Pat. No. 5,116,662 issued May 26, 1992 to Morman, which are incorporated herein by reference. A two-directional stretch material may comprise a composite material including a stretchable material and an elastic sheet, which may be formed by meltblowing or extrusion. Neckable materials are those which may be constricted in at least one dimension by applying a tensioning force in a direction perpendicular to the desired direction of neck down, and may include a spunbonded, meltblown or bonded carded web. The tensioned, necked neckable material may be joined to the elongated elastic sheet at spaced locations arranged in a nonlinear configuration. Another two-directional stretch composite material may comprise one or more layers of reversibly necked material joined to one or more layers of elastic sheet at spaced locations. Reversibly necked materials are those that have been treated, such as with heat, while necked to impart memory to the material so that, when a force is applied to extend the material to its pre necked dimensions, the treated, necked portions will generally recover to their necked dimensions upon termination of the force.

The elastic side panels may also be made of the type of material known as “stretch bonded,” which typically refers to an elastic member being bonded to another member while the elastic member is extended at least about 25 percent of its relaxed length.

The components of the chassis may be held together by adhesives ultrasonic bonding, heat, pressure or another technique known to the art. The backing member should provide a liquid barrier and optimally should be breathable. It may also be covered with an additional layer or layers of material, an outer member (not shown in the Figures), to further enhance or change the outside appearance, feel, or functionality of the outside of the pant. The absorbent pad is that portion of the pant that absorbs and retains liquids. It may be made of any absorbent material, such as that known to the art for use in diapers, adult incontinence products and feminine protection products, which would include porous fibrous matrices or foams. Super absorbent additives such as those made by DOW (e.g. Dow 2035) and Favor (e.g. SXM880) may also be added to the pad. The thickness and absorbtivity of the pad may vary depending on product design and intended use from very thin to very thick, more specifically current pads may vary in thickness from about 1 mm to about 25 mm.

The pad may be shaped in any number of different configurations known to the art. It may be hourglass shaped, rectangular, oval or it may be some other less regular shape, such as trapezoidal. The pad may extend length wise over the entire length of the chassis or it may be shorter. The pad may similarly extend the entire width of the chassis or it may be narrower. Additionally, the pad may have surface contours in it such as, for example and without limitation, a depression in the center, multiple depressions or it may be embossed. In addition, the absorbent pad may comprise multiple layers of materials providing desired intake distribution and retention functionality's.

The pad may be covered by a porous wrap. For example, this porous wrap may be C-fold of tissue paper or other open material. Ideally, the porous wrap is hydrophilic and has wet strength. By C-fold what is meant is that the material wraps, or is folded, around the pad but does not completely surround it, thus leaving the material in the shape of a C. The material may also, however, completely encompass the pad or be located on only the top and/or bottom of the pad. The material may either be glued or not glued to the pad. Ideally, when tissue is used it is not glued or otherwise adhered to the pad. The surge layer, which is optional, is placed on the bodyside of the pad.

The surge layer may be adhered to the tissue, the pad, bodyside liner or any combination thereof. Ideally, it is adhered to the tissue or pad.

The pad is held in place in the chassis by being affixed to the backing member, the bodyside liner, or both, or by the sandwiching effect of the backing member and bodyside liners, or both of these mechanisms. The bodyside liner may be any liquid permeable sheet. Optimally, the bodyside liner is a spunbonded polypropylene. The bodyside liner may be adhered to the surge layer, or the tissue if there is no surge layer, or the pad if there is no surge layer and no tissue, by way of adhesives (which are well known to the art and some of which have been expressly identified herein). The adhesive may be applied by any of the ways known to the art, some of which have been described herein. The pad may similarly be affixed to the backing member. The backing member may be any water resistive sheet. Optimally, the backing member will be a water impervious film that is also breathable. The backing member may be adhered to the pad. The backing member and the bodyside liner should be affixed to each other around the periphery of the pad.

Spunbonded fibers are typically small diameter fibers which are formed by extruding molten thermoplastic materials as filaments from a plurality of fine, usually circular capillaries of a spinneret with the diameter of the extruded filaments then being rapidly reduced as by, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartman, and U.S. Pat. No. 3,542,615 to Dobo et al. Spunbond fibers are generally not tacky when they are deposited onto a collecting surface. Spunbond fibers are generally continuous and have average diameters (from a sample of at least 10) larger than 7 microns, more particularly, between about 10 and 20 microns.

Meltblown fibers are typically fibers of polymeric materials which are generally formed by extruding a molten thermoplastic material through a plurality of fine, usually circular, die capillaries as molten threads or filaments into converging high velocity, usually hot, gas (e.g. air) streams which attenuate the filaments of molten thermoplastic material to reduce their diameter. Thereafter, the meltblown fibers can be carried by the high velocity gas stream and are deposited on a collecting surface to form a web of randomly dispersed meltblown fibers. Such a process is disclosed, for example, in U.S. Pat. No. 3,849,241 to Butin et al. Meltblown fibers may be continuous or discontinuous, are generally smaller than 10 microns in average diameter, and are generally tacky when deposited onto a collecting surface.

Breathable members have a water vapor transmission rate (WVTR) that is equal to or greater than 500 g/m²124 hours. Ideally, breathable members have WVTR of equal to or greater than 1000 g/m²/24 hours. Optimally, breathable members have WVTR of equal to or greater than 2000 g/m²124 hours. Such materials may be used, for example as a backing member. Materials having a WVTR as high as 8,000 g/m²/24 hours or greater may be used for side panels. The WVTR is calculated in accordance with ASTM Standard E96-95. Thus, circular samples measuring three inches in diameter are cut from each of the test materials and from a control, which is a piece of CELGARD™ 2500 film from Hoechst Celanese Corporation of Sommerville, N.J. CELGARD™ film is a microporous polypropylene film. Three samples are prepared for each material. The test dish is number 60-1 Vapometer pan distributed by Thwing-Albert Instrument Company of Philadelphia, Pa. One hundred milliliters of water is poured into each Vapometer pan and individual samples of the test materials and control materials are placed across the open tops of the individual pans. Screw-on flanges are tightened to form a seal along the edges of the pan, leaving the associated test material or control material exposed to the ambient atmosphere over a 6.5 centimeter diameter circle having an exposed area of approximately 33.17 square centimeters. The pans are placed in a forced air oven at 100° F. (32° C.) or 1 hour to equilibrate. The oven is a constant temperature oven with external air circulating through it to prevent water vapor accumulation inside. A suitable forced air oven is, for example, a Blue M Power-O-Matic 60 oven distributed by Blue M. Electric Company of Blue Island, Ill. Upon completion of the equilibration, the pans are removed from the oven, weighed an immediately returned to the oven. After 24 hours, the pans are removed from the oven and weighed again. The preliminary test water vapor transmission rate values are calculated with Equation (1) below:

Test WVTR=(grams weight loss over 24 hours)×315.5 g/M²/24 hours. The relative humidity within the oven is not specifically controlled. (I)

Under the predetermined set conditions of 100 F (32 C) and ambient relative humidity, the WVTR for the CELGARD™ 2500 control has been defined to be 5000 grams per square meter for 24 hours. Accordingly, the control sample is run with each test and the preliminary test values are corrected to set conditions using Equation (II) below:

WVTR=(Test WVTR/control WVTR)×(5000 g/m²124 hours). (II)

Further embodiments and constructions of the body absorbent pad and chassis components that may be used are as follows. These embodiments are provided as examples and are made without limitation.

The absorbent pad comprises materials adapted to absorb and retain urine, menses, blood, or other body excrement. The absorbent pad may comprise various natural or synthetic absorbent materials, such as cellulose fibers, surfactant treated meltblown fibers, wood pulp fibers, regenerated cellulose or cotton fibers, a blend of pulp and other fiber, or the like. The absorbent pad may be of a generally rectangular shape and includes a peripheral edge comprised of side edges, a front end edge, and a back end edge. The absorbent pad has an exterior surface that faces away from the wearer, and an interior surface that faces towards the wearer.

The porous fibrous matrix of absorbent pad is preferably an air laid bat of fluff and high absorbency material which may be formed in many ways, for example according to the teaching of Mazurak and Fries as set forth in U.S. Pat. No. 4,381,782, the disclosure of which is incorporated herein by reference. The absorbent pad can comprise an airformed mixture of high absorbency material (SAP) and fibers, preferably of fluff pulp. Most preferably, the mixing of the fluff fibers and the high absorbency material is homogenous. Alternatively, the mixtures can be layered, phased to place the high absorbency material in a specific machine direction location, or placed in a narrow band in the cross direction, or both. Also, the fibers can be made from fibers other than fluff pulp such as chemically stiffened and thermomechanical pulps. In addition, the absorbent pad can comprise absorbent material other than air formed fluff and SAP. For example, coform materials as referenced in U.S. Pat. No. 4,818,464 to Lau and U.S. Pat. No. 4,100,324 to Anderson can be used to make the absorbent pad as long as they also contain high absorbency materials. In addition, wet formed composite materials comprising a combination of fibers and high absorbency materials as disclosed in U.S. Pat. No. 5,651,862 to Anderson et al. can also be used. Stabilized airlaid materials comprising a mixture of fibers, binder fibers, and high absorbency materials which are bound together by latex binding or through air bonding are also usable as absorbent materials. Additionally, any material known in the art that serves to absorb body exudates can be used to construct the absorbent pad.

The absorbent pad may comprise high absorbency materials which are typically hydrogel polymers that are desirably sufficiently cross-linked to render the materials substantially water-insoluble. Cross-linking may, for example, be by irradiation or by covalent, ionic, van der Waals or hydrogen bonding. Suitable materials are available from various commercial vendors, such as Dow Chemical Company (Drytech 2035 LD), Stockhauser, Inc., Hoechst-Celanese Corporation and Allied-Colloid. Typically, the high absorbency material is capable of absorbing at least about 15 times its weight in water, and desirably is capable of absorbing more than about 25 times its weight in water. These high-absorbency materials may further include organic and inorganic materials. Organic high-absorbency materials can include natural materials, such as pectin, guar gum and peat moss, as well as synthetic materials, such as synthetic hydrogel polymers. Such hydrogel polymers may include, for example, carboxymethylcellulose, alkali metal salts of polyacrylic acids, polyacrylamides, polyvinyl alcohol, ethylene maleic anhydride copolymers, polyvinyl ethers, hydroxypropyl cellulose, polyvinyl morpholinone, polymers and copolymers of vinyl sulfonic acid, polyacrylates, polyacrylamides, polyvinyl pyridine or the like. Other suitable polymers can include hydrolyzed acryolonitrile grafted starch, acrylic acid grafted starch, and isobutylene maleic anhydride copolymers, and mixtures thereof. Suitable high-absorbency materials are described in U.S. Pat. No. 4,699,823 issued Oct. 13, 1987 to Kellenberger et al. and U.S. Pat. No. 5,147,343 issued Sep. 15, 1992 to Kellenberger, which are incorporated herein by reference.

The high-absorbency material can be distributed or otherwise incorporated into the absorbent pad employing various techniques. The high absorbency material can be substantially uniformly distributed among the fibers comprising the absorbent pad. The materials can also be non-uniformly distributed within the fibers of the absorbent pad to form a generally continuous gradient with either an increasing or decreasing concentration of high-absorbency material, as determined by observing the concentration moving inward from the backing member. Alternatively, the high-absorbency material can comprise a discrete layer separate from the fibers of the absorbent pad, or can comprise a discrete layer integral with the absorbent pad.

The absorbent pad may also include a porous wrap or wrap layer to help maintain the integrity of the fibrous absorbent pad. This wrap layer may comprise a cellulosic tissue or spunbond, meltblown or bonded-carded web material composed of synthetic polymer filaments, such as polypropylene, polyethylene, polyesters or the like or the like or natural polymer filaments such as rayon or cotton.

The absorbent pad may have an aqueous liquid capacity great enough to absorb discharges from about 10 grams or less to about 1500 grams or more. The absorbent pad may have a thickness less than about 25 mm, thus providing a non-bulky and flexible fit. For an adult incontinence product, the capacity of the absorbent pad should have a total capacity of about 200 grams to about 1300 grams. The absorbent pad may have a total capacity of at least about 300 grams and not more than about 1200 grams. Further, the total capacity of the absorbent pad may be from about 400 grams to about 800 grams.

The total capacity of the absorbent pad is determined using the absorbent pad, the bodyside liner, the backing member or outer liner, and the outer member. The saturated retention capacity is a measure of the total absorbent capacity of an absorbent garment. The saturated retention capacity is determined as follows. The absorbent garment to be tested, having a moisture content of less than about 7 weight percent, is weighed and submerged in an excess quantity of the room temperature (about 23° C.) saline solution described below. The material is allowed to remain submerged for 20 minutes. After 20 minutes the absorbent garment is removed from the saline solution and placed on a Teflon TM coated fiberglass screen having 0.25 inch openings (commercially available from Taconic Plastics Inc., Petersburg, N.Y.) which, in turn, is placed on a vacuum box and covered with a flexible rubber dam material. A vacuum of 3.5 kilopascals (0.5 pounds per square inch) is drawn in the vacuum box for a period of 5 minutes. The absorbent garment is weighed again. The amount of aqueous liquid retained by the material being tested is determined by subtracting the dry weight of the absorbent garment from the wet weight of the absorbent garment (after application of the vacuum) and is reported as the saturated retention capacity in grams of aqueous liquid retained.

The saline solution is an aqueous solution of about 0.9 percent sodium chloride by weight. A suitable product is S/p™ Certified Blood Saline commercially available from Baxter Diagnostics in McGaw Park, Ill.

The absorbent pad may also include tissue layers or acquisition or distribution layers to help maintain the integrity of fibrous absorbents or transport aqueous liquids. Thus, the body may also include additional components to assist in the acquisition, distribution, and storage of body exudates. For example, the body may include a transport layer, such as described in U.S. Pat. No. 4,798,603 issued Jan. 17,1989 to Meyer et al., or a surge management layer, such as described in U.S. Pat. No. 5,486,166 issued Jan. 23,1996 to Bishop et al., U.S. Pat. No. 5,364,382 issued Nov. 15,1994 to Latimer et al., U.S. Pat. No. 5,490,846 to Ellis et al., U.S. Pat. No. 5,429,629 to Latimer et al., U.S. Pat. No. 5,509,915 to Hanson et al., U.S. Pat. No. 5,192,606 to Proxmire et al., an European Patent Application EP 0 539 703 A1, published May 5, 1993, which the patents and application are incorporated herein by reference. Such layers are also referred to as acquisition/distribution or surge layers.

The acquisition/distribution layer is disposed on the aqueous liquid storage layer toward the body-facing surface of the absorbent pad to help decelerate and diffuse surges of aqueous liquid that may be introduced into the absorbent pad. The acquisition/distribution layer may comprise a through air bonded carded web composed of a blend of 40% 6 denier polyester fibers, commercially available from Hoechst Celanese Corporation, and 60% 3 denier polypropylene/polyethylene sheath core bicomponent fibers, commercially available from the Chisso Corporation, with an overall basis weight ranging of from about 50 gsm and to about 120 gsm.

One suitable absorbent pad comprises an aqueous liquid storage layer in which the basis weight of the absorbent components, such as fluff, pulp, and superabsorbent (SAP), are generally continuous throughout the MID length of the absorbent pad. The distribution of the absorbent components is substantially homogenous in at least the y-direction, preferably in the x- and y directions and may be homogenous in the z-direction within the absorbent pad. The basis weight of the absorbent pad can range between about 80 gsm and about 1,000 gsm. An acquisition layer is disposed on the aqueous liquid storage layer, which is typically moved forward on the aqueous liquid storage layer. The fluff pulp/SAP ratio can range from about 100:0 to about 40:60, and more typically from about 80:20 to about 50:50.

One suitable absorbent pad provides the feature of being able to transport aqueous liquid in what can be characterized in an x- and y-direction and in a z-direction. The transport of aqueous liquid in the z-direction is movement of a wicking nature and gravity flow where the aqueous liquid moves away from the body of the wearer. The transport of aqueous liquid in the x-direction and y-direction is movement and/or wicking of aqueous liquid along the length and width of the absorbent pad. As can be appreciated, the movement of aqueous liquid both away from the wearer and along the length and width of the absorbent pad results in an increase in the utilization of the area of the absorbent pad since the aqueous liquid moves towards the distal ends of the absorbent pad, and the result is an improvement of the absorption characteristics of the absorbent pad.

The backing member prevents aqueous liquid strike through to the outer clothing when discharge occurs onto the absorbent pad. The backing member is located on the inside of the outer member, if an outer member is used, and typically consists of an aqueous liquid impervious film such as polyethylene. The aqueous liquid impervious backing member has an exterior surface that faces away from the wearer and an interior surface that faces toward the wearer. The backing member, acting as a barrier, should retard the movement of the aqueous liquid through the garment by making the backing member resistant to penetration normally encountered under wearing conditions. The backing member desirably comprises a material that is formed or treated to be aqueous liquid impervious. Alternatively, the backing member may comprise an aqueous liquid pervious material and other suitable means, such as an aqueous liquid impervious layer associated with the absorbent pad may be provided to impede aqueous liquid movement away from the absorbent pad. The pant may be rendered aqueous liquid impervious by any method well known in the art such as coating the absorbent pad or by securing a separate aqueous liquid impervious material to the absorbent pad. The backing member may comprise a thin, aqueous liquid impervious web or sheet of plastic film such as polyethylene, polypropylene, polyvinyl chloride or similar material. Other acceptable materials include a single spunbonded layer of the above types of materials, spunbonded-meltblown-spunbonded material. Suitable foam materials may also be used, as well as materials that are both aqueous liquid impervious and vapor-pervious.

Alternately, the backing member may comprise a nonwoven, fibrous web which has been suitably constructed and arranged to have low aqueous liquid perviousness. Still alternately, the backing member may comprise a layered or laminated material, such as a thermally bonded plastic film and nonwoven web composite. Alternatively, the backing member consists of an aqueous liquid impervious film or foam which is pervious to water vapor under normal wearing conditions. The backing member may have a water vapor transmission rate of at least about 800 grams/m²/24 hours measured by ASTM E96-92. One example of a suitable film is a 39.4 grams per square meter microporous film produced by Mitsui and sold by Consolidated Thermoplastics (CT) under the trade name of ESPOIR® N-TAF-CT.

The outer member may be compliant and soft feeling to the wearer. The outer member may be any soft, flexible, porous sheet which is aqueous liquid pervious, permitting aqueous liquids to readily penetrate into its thickness, or impervious, resistant to the penetration of aqueous liquids into its thickness. A suitable outer member may be manufactured from a wide range of materials, such as natural fibers (e.g., wood or cotton fibers), synthetic fibers (e.g., polyester or polypropylene fibers) or from a combination of natural and synthetic fibers or reticulated foams and apertured plastic films.

There are a number of manufacturing techniques which may be used to manufacture the outer member. For example, the outer member may be woven or nonwoven web or sheet such as a spunbond, meltblown or bonded carded web composed of synthetic polymer filaments, such as polypropylene, polyethylene, polyesters or the like, or a web of natural polymer filaments such as rayon or cotton. The bonded-carded web may be thermally bonded or sprayed with a binder by means well known to those skilled in the fabric art. Suitably, the outer member is a nonwoven spunbond material and is available from Kimberly-Clark Corporation, located in Roswell, Ga. The outer member has a weight from about 0.3 oz. per square yard (osy) (10.2 gsm) to about 2.0 osy (68 gsm) and typically about 0.6 osy (20.4 gsm). The outer member of the garment may be printed, colored or decoratively embossed. The outer member preferably has a pore size that readily allows the passage there through of air, sweat, and perspiration due to the breathability of the materiality. The outer member may be selectively embossed or perforated with discrete slits or holes extending therethrough.

The pant may further include an aqueous liquid pervious bodyside liner that may be of approximately the same dimension as the back member and has an aqueous liquid peripheral edge comprising a front edge, a back edge, and side edges. The aqueous liquid pervious bodyside liner has an exterior surface that faces away from the wearer and an interior surface that faces towards the wearer.

The bodyside liner consists of a nonwoven or other soft material for contacting the wearer's skin. The bodyside liner is compliant and soft feeling to the wearer. The bodyside liner may be any soft, flexible, porous sheet which is aqueous liquid pervious, permitting aqueous liquids to readily penetrate into its thickness. A suitable bodyside liner may be manufactured from a wide range of materials, such as natural fibers (e.g., wood or cotton fibers), synthetic fibers (e.g., polyester or polypropylene fibers) or from a combination of natural and synthetic fibers or reticulated foams and apertured plastic films.

The bodyside liner is formed of an aqueous liquid pervious material so that aqueous liquid waste, and possibly semi-solid waste as well, can pass through to the absorbent pad and be absorbed by the absorbent pad. A suitable bodyside liner may be comprised of a nonwoven web, a spunbond, meltblown or bonded-carded web composed of synthetic polymer filaments or fibers, such as polypropylene, polyethylene, polyesters or the like, a perforated film, or a web or natural polymer filaments or fibers such as rayon or cotton. In addition, the bodyside liner may be treated with a surfactant to aid in aqueous liquid transfer. Suitably, the bodyside liner is a nonwoven spunbond. Suitably, the spunbond material is available from Kimberly-Clark Corporation, located in Roswell, Ga. The bodyside liner has a weight from about 0.3 oz. (10.2 gsm) per square yard (osy) to about 2.0 osy (68 gsm) and typically about 0.5 osy (17 gsm). The bodyside liner may be printed, colored or decoratively embossed. The bodyside liner can also be a nonwoven web or sheet of polymeric fibers, such as polypropylene, polyester, polyethylene, rayon, chisso and the like. The bodyside liner may also be a plastic film with perforations, an expanded plastic webbing material or a scrim material. The bodyside liner has a pore size that readily allows the passage therethrough of air, sweat, and perspiration due to the breathability of the material. The bodyside liner may be selectively embossed or perforated with discrete slits or holes extending therethrough.

Ideally, the fabric of the bodyside liner is surface treated with a surfactant such as that commercially available from Union Carbide Chemicals and Plastics Company, Inc., of Danbury, Conn., U.S.A., under the trade designation TRITON X-102. As used herein, the term “fabric” refers to all of the woven, knitted and nonwoven fibrous webs. The term “nonwoven web” means a web of material that is formed without the aid of a textile weaving or knitting process.

As an alternate material, an aqueous liquid pervious bodyside liner can be made of a carded web of polyester fibers bonded to a spunbonded polypropylene or polyethylene carrier sheet. The carded material is made up of about 20 to about 60 weight percent polypropylene or polyethylene and about 80 to about 40 weight percent polyester. The basis weight of this material can be between about 30 gsm and about 70 gsm.

The crotch elastics are sandwiched in-between the backing member and bodyside liners. Alternatively, the crotch elastics may be sandwiched in between the backing member and outer member. They may be held in place by adhesives, ultrasonic bonding, heat, pressure or any other technique known to the art for holding elastic in place. Optimally, by way of example and without limitation they may be held in place by an adhesive placed in a spiral pattern. The elastics may be made from any material known in the art, such as LYCRA® from Dupont, Wilmington, Del. or GLO SPAN from Globe Manufacturing Company, Fallriver, Mass. The elastics may be in ribbon form, sheet form, string form, yarn form, or any other type. They may also be heat activated. Ideally, they consist of 3 strands of LYRCA® having a decitex of about 940. Although 3 strands of crotch elastic are shown in the Figures, it is understood that more or less may be used. Further examples of the types of elastics and the manner in which they are affixed are provided in Heran et al. U.S. Pat. No. 4,642,362, Strohbeen et al. U.S. Pat. No. 4,610,681 and Ales et al. U.S. Pat. No. 4,639,949, the disclosures of which are herein incorporated by reference. Further, the crotch elastics may extend along the leg cut out and into the area of the back of the chassis. In this way, crotch elastics may extend to or past the point where the side panels are joined to the chassis.

Further embodiments and constructions of the crotch elastics are as follows. These embodiments are provided as examples and are without limitation. In an embodiment, elastics are attached to the pant sandwiched between the backing member and the bodyside liner, in generally a stretched state by means known in the art, including ultrasonically bonded, heat/pressure bonded or adhesively bonded. The elastics are typically attached in a stretched state by means known in the art, including ultrasonically bonded, heat/pressure bonded or adhesively bonded. Materials suitable for the elastics include a wide variety including but not limited to elastic strands, yarn rubber, flat rubber, elastic tape, film-type rubber, polyurethane and elastomeric, tape-like elastomeric or foam polyurethane or formed elastic or non-elastic scrim. Suitable material is sold under the name LYCRA® by the DuPont Company located in Wilmington, Del. Each elastic may be unitary, multi-part or composite in construction before integrating into the garment.

In an alternative embodiment, elastics are attached to the garment sandwiched between the outer member and the backing member in generally a stretched state by means known in the art, such as ultrasonically bonded, heat/pressure bonded or adhesively bonded.

The elastics may be from about 0.0625 inch (1.6 mm) to about 1 inch (25 mm) wide, more typically from about 0.25 inch (6 mm) to about 1 inch (25 mm), and most typically from about 0.25 inch (6 mm) to about 0.75 inch (18 mm) such as 0.5 inch (13 mm). The elastics are applied under an elongation of from about 100% to about 350%, more typically under an elongation of from about 150% to about 275%, and most typically under an elongation of from about 225% to about 275%.

The elastics may comprise threads, strands, ribbons, bands, film, elastic nonwovens, or composite. The threads, strands, ribbons, or bands may be multiple and may be applied as a composite. The number of pieces of elastic material comprising the elastic ranges from about 1 to about 6, more typically from about 2 to about 5, and most typically from about 3 to about 4. Preferably, when the elastics are threads, 1 to 6 threads are used as the leg elastics, and the threads are spaced from about 0.0625 inch (1.6 mm) to about 0.5 inches (13 mm), more preferably from about 0.0625 inch (1.6 mm) to about 0.25 inch (6 mm), and most preferably about 0.125 inch (3 mm) apart.

The threads may be made of any suitable elastomeric material. One suitable material is spandex such as LYCRA® threads available from DuPont located in Wilmington, Del. Suitable elastics include threads having a decitex (g/10000 m) of from about 470 to about 1200, more typically from about 620 to about 1000, and most typically from about 740 to about 940 for elastics comprising from about 3 to about 6 threads. Adhesive, typically applied in a meltblown or swirl pattern using currently known technology, is used to bond the elastics to the outer member, the bodyside liner or the backing member. Preferably the adhesive is applied only to the elastics. A suitable adhesive includes, for example, Findley H2096 hot melt adhesive which is available from Ato Findley Adhesives located in Milwaukee, Wis.

The cuffs may be made from any of the types of material that the bodyside liner or outer liner are made from or they may be made from some other type of material. The cuffs may be adhered to the bodyside liner by adhesives, ultrasonic bonding, heat, pressure or any of the other bonding techniques know to the art, of which many are disclosed herein. The cuffs may be formed from the bodyside liner. The cuffs may have elastic in them on either or both the bodyside of the cuff, the side of the cuff that is adhered to the liner, the center of the cuff or on any other locations. The elastics may run the length of the cuff and the pant or they may be shorter. The cuffs may be the entire length of the chassis or they may be shorter. They may run in a straight line, as shown in FIG. 1, or they may be curved either to follow the contour of the leg cut out shape or to not follow that contour. They may be the same length, longer, or shorter than the length of the core. Ideally, the cuffs are straight, made from the same material as the bodyside liner, have elastics in them that are positioned adjacent the bodyside, i.e., away from the points where they are bonded to the liner and that run the entire length of the cuff, and extend length wise to just beyond the ends of the core.

The bodyside liner, the outer liner, and the cuff material, as well as any other materials in the pant may be embossed or otherwise patterned by ultrasonics, pressure, or heat.

An elastic waistband may optionally be incorporated into the pant at the front end, at the back end, or at both ends. The elastic waist band may be made from any elastic material known to the art, such as for example a composite of spunbonded polypropylene and elastic strands. Ideally, the waistband is included in the back section of the chassis and is made from the same type of material as the elastic side panels, although other types of elastized materials may also be used. The waistband may be positioned on or between any of the layers of the chassis. The waistband may be held in place in the pant by any of the techniques known to the art, many of which have been disclosed herein.

The front fastening panel may be made from any material that has a surface that will adhere to the surface of the closure devices. Such closure devices are well known to the art and may include by way of example and without limitations, mechanical fasteners; adhesive tapes; adhesive tabs; buttons; ties; Velcro®; hooks; clips; snaps; hook and loop; bottom and loop; interlocking hook; hook and hook type devices. A preferred material for the front fastening panel and one that is readily refastenable to hook closure devices is the material described in the co-pending Stokes U.S. pat. application Ser. No. 754,419, filed Dec. 16, 1996, which is assigned to Kimberly-Clark, and which is incorporated herein by reference.

The general configuration of this pant provides for many variations and alternative embodiments, which gives rise to many variations and alternatives in the wearing, use and fit of the pant.

The entire outer edge of one or both of the elastic panels, i.e., the edge to which the closure devices are attached, may be stiffened. This stiffening may be accomplished by any method known to the art, such as by ultrasonics, heat, glue or the use of additional material. This stiffened portion would have reduced elasticity or be non-elastic. A pant with the stiffened outer panel edge conforms to the fastening panel and lies flat against the body and prevents gapping between the closure devices on the panels.

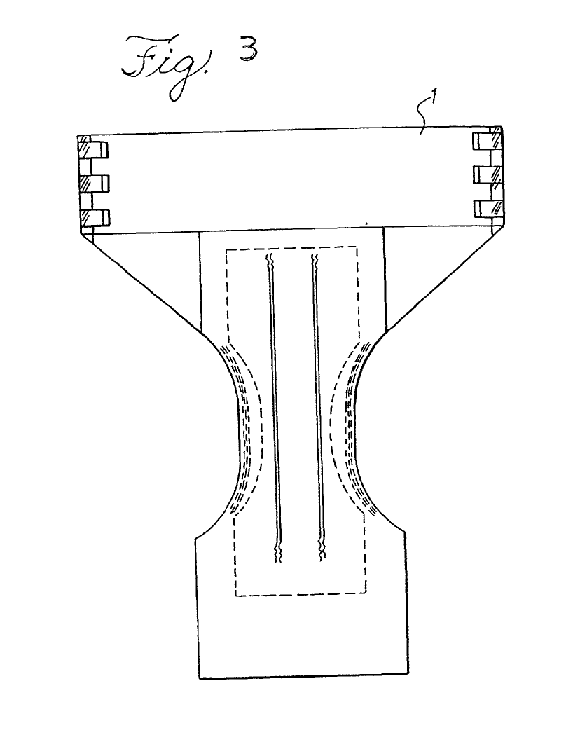

On the outside of the absorbent pad there may be a backing member 16, which may cover the absorbent pad. Further details of the construction and composition of such products are disclosed in co-pending United States patent applications filed on Dec. 18, 1998 in the names of Datta et al., titled “Refastenable and Adjustable Pant” (Ser. No. 09/215,866) and in the names of Fell et al., titled “Stretchable Composite Material Having Continuous Gathers” (Ser. No. 09/215,951), which are assigned to Kimberly-Clark Corporation, and the disclosures of which are both incorporated herein by reference. In another example of the pant, the elastic side panels may be a single elastic panel that extends across the entire back of the product. A product is provided having a general configuration of the type shown in FIG. 3. In this example, the elastic panels are joined together to form a single elastic panel 1 attached to the chassis. This single panel can be viewed as having two portions, a left and a right divided along the center line of the pant.

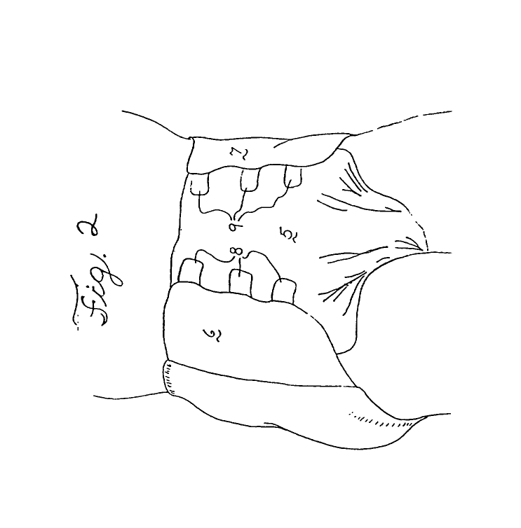

In use, the pant is positioned on the user and fastened by securing the closure devices 8 and 9 to the front fastening panel 5, as is shown in FIG. 2. When the pant has been soiled, it is removed from the user and it can be folded into a convenient pouch by folding or rolling the front side 2 towards the back and by also folding or rolling one of the elastic side panels in. The remaining elastic side panel is then wide enough and has sufficient elasticity to encircle the entire rolled up configuration and close onto itself creating a convenient pouch for disposal. Because of the width of the elastic panel, it will impart elastic properties to the pouch. Moreover, the use of a single elastic panel to encircle the product making a pouch increases the ease of handling the product for disposal purposes. It creates a discrete package for disposal. Also, if the elastic panel has pleats or other structure, it provides better grip making the pouch easier to handle.

For an adult product, the pant may be able to retain about 100 to 1,500 grams of bodily waste (i.e., urine, fecal matter, etc.). For an infant or child, the pant may be able to retain from about 25 to about 250 grams of such material. Thus, the relationship of the size, and elasticity of the elastic panels, as well as the number and position of the closure devices, provides the ability to have a single panel wrap around a used adult or infant product. By used it is meant that the product has at least the minimum amount bodily waste it is capable of absorbing and holding while the user is wearing it. By fully loaded it is meant that the product has the maximum amount of bodily waste it is capable of absorbing and holding while the user is wearing it.

Having the ability to have a single elastic panel wrap around the entire used product (as well as a fully loaded product) to form a disposal pouch greatly decreases the chances of leaks or spills. Additionally, when the panel wrapping a used product is elastic, it may retain some, if not a substantial amount, of its elastic properties when wrapped around the product. In this instance the pouch will also have elastic properties. These elastic properties of the pouch also help prevent leakage during disposal, for example by providing extra give, or resilience, if the pouch is dropped or crushed. Moreover, the elasticity of the panels provides a pouch that can better hold and retain solid bodily waste from shifting or falling from the pouch.

1. A pant comprising a chassis and a pair of elastic panels; the pant having a front and back end; the chassis comprising an absorbent core, a backing member and a bodyside liner; the chassis having a length and a width and a front and a back end; the front end having a fastener; the elastic panels having a width and a length; the elastic panels being elasticized along the majority of their length and width; the elastic panels adjacent the chassis and toward the back end of the chassis; and, the width of at least one of the elastic panels being such that the panel when stretched can encircle the pant when used.

2. The pant of claim 1 wherein on elastic panel is wide enough to encircle the used pant at least 1½ times.

3. An incontinence product for adults comprising: a chassis comprising an absorbent core and a refastenable elastic side panel; the elastic side panel having a length and a width; the length and the width of the elastic side panel being substantially the same; the elastic side panel being elasticized along the majority of its length and width; the chassis having a length and a width; and the width of the elastic side panel being at least long enough to surround the product when used and fasten to itself, thereby forming an elastic disposable pouch.

4. The product of claim 3 wherein the used product can hold at least about 100 grams of bodily waste.

5. The product of claim 3 wherein the used product can hold at least about 400 grams of bodily waste.

6. A pant product for toddlers and infants comprising: a chassis comprising an absorbent core and a refastenable elastic side panel; the elastic side panel having a length and a width; the elastic side panel being elasticized along the majority of its length and width; the chassis having a length and a width; the length of the elastic side panel being at least about 25% of the length of the chassis; the width of the elastic side panel being at least long enough to fully surround the product when used and fasten to itself, thereby forming an elastic disposable pouch.

7. The product of claim 6 wherein the used product can hold at least about 25 grams of bodily fluid.

8. The product of claim 6 wherein the used product can hold at least about 100 grams of bodily fluid.

9. A pant product comprising a chassis and one elastic back panel; where the back panel can be separated into a left side portion and a right side portion by the centerline of the product; each side portion having a length and a width; the product having a length and a width; the width of one of the elastic side portions being at least long enough to encircle the product when used and fasten to itself, thereby forming an elastic disposable pouch.

10. An absorbent product comprising: a chassis; an absorbent core; two discrete refastenable elastic side panels; each elastic panel having a length and a width; the length and width of each elastic panel being substantially the same; the chassis having a length and a width; and, the width of each elastic panel being at least long enough to surround the product when used and fasten to itself, thereby forming an elastic disposable pouch.

11. The product of claim 10, wherein the elastic panels are permanently attached to the chassis.

12. An absorbent product comprising: a chassis; an absorbent core; two discrete refastenable elastic side panels; each elastic panel having a length and a width; the elastic panels permanently attached to the chassis; the chassis having a length and a width; and, the width of each elastic panel being at least long enough to surround the product when used and fasten to itself, thereby forming an elastic disposable pouch.

13. The product of claim 12, wherein the length and width of each elastic panel being substantially the same.

14. An absorbent product comprising: a chassis; an absorbent core; two discrete refastenable elastic side panels; each elastic panel having a length and a width; the elastic panels permanently attached to the chassis; the length and width of each elastic panel being substantially the same; the chassis having a length and a width; and, the width of each elastic panel being at least long enough to surround the product when used and fasten to itself, thereby forming an elastic disposable pouch.
